(19) United States
(12) Patent Application Publication
(10) Pub. No.: US 2008/0008716 A1
(43) Pub. Date: Jan. 10, 2008

(54) Combined Pharmaceutical Composition Comprising an Anti-4-1BB Monoclonal Antibody and Chemotherapeutic Anti-Cancer Agent for Preventing and Treating Cancer Disease

(75) Inventor: Byoung Se KWON, Ulsan (KR)

Correspondence Address: KIRK HAHN, 14431 HOLT AVE, SANTA ANA, CA, 92705, US

(73) Assignee: ULSAN INDUSTRIAL EDUCATION FOUNDATION, Ulsan (KR)

(21) Appl. No.: 11/772,806

(22) Filed: Jul. 2, 2007

(30) Foreign Application Priority Data
Jul. 4, 2006 (KR) 10-2006-62400

(51) Int. Cl. A61K 39/395; A61P 35/00

(52) U.S. Cl. 424/158.1

(57) ABSTRACT

A combined composition comprising an anti-4-1BB monoclonal antibody and chemotherapeutic anti-cancer agent for preventing and treating cancer disease. Provides a pharmaceutical composition comprising the combined mixture of anti-4-1BB antibody and chemotherapeutic anti-cancer agent enhancing the specific immune response to cancer and killing cancer cell for treating or preventing cancer disease as an effective ingredient, together with a pharmaceutically acceptable carrier.

### BACKGROUND OF THE INVENTION

A lot of inventors have made efforts to develop methods and medicines for treating cancer disease that overcome the side effects of chemotherapy and radiotherapy. Because of various advanced biological techniques involved in cancer and immune cells, new methods of treating cancer or immune diseases have not been designed till now. One of the interesting methods is immunotherapy, which enhances the immune response or reduces the immune tolerance against tumors to increase the anti-cancer immune response (Waldmann T A, Nat. Med. Rev., 9, pp 269˜277, 2003; Ye Z et al., Nat. Med., 8, pp 343˜348, 2002; Yu P et al., Nat. Immunol., 5, pp 141˜149, 2004).

In the method for treating cancer disease, there have been reported that the potency of agonistic anti-4-1BB antibody has already been demonstrated and has been mediated by increasing natural killer cell, CD8+ T cell activity and IFN-γ (Xu D et al., Int. J. Cancer, 109, pp 499˜506, 2004; Ito F et al., Cancer Res., 64, pp 8411˜8419, 2004; Sun Y et al., J. Immunol., 168, pp 1457˜1465, 2002; Ju S A et al., Immunol. Cell Biol., 83, pp 344˜351, 2005). However, it has been reported that the sole medication of anti-4-1BB antibody was not sufficient to completely inhibit the growth of melanoma (Ju S A et al., Immunol. Cell Biol., 83, pp 344˜351, 2005).

There have been reported that agonistic anti-4-1BB antibody preferentially induces the response of CD8+ T cell, thus 4-1BB has been investigated as one of prominent candidate to treat cancer disease for a long time (Shuford W W et al., J. Exp. Med. 186(1), pp 47˜55, 1997; Halstead E S et al., Nat. Immunol., 3(6). pp 536˜541, 2002). Although the previous studies showed that the treating method of cancer disease using by 4-1BB antibody had been successful, the sole medication of agonistic anti-4-1BB antibody have been ineffective to treat the poorly immunogenic tumors (Chen S H et al., Mol. Ther., 2(1), pp 39˜46, 2000; Martinet O et al., Gene Ther., 9(12), pp 786˜792, 2002; Melero I et al., Nat. Med., 3(6), pp 682˜685, 1997; Wilcox R A et al., J. Clin. Invest., 109(5), pp 651˜659, 2002; Ju S A et al., Immunol. Cell Biol., 83(4), pp 344˜351, 2005). In case of cancer cell showing low immunity, T cell activation is difficult, so the effect of anti-4-1BB antibody becomes inefficient with the low frequency of cells expressing 4-1BB. To overcome the limit of anti-caner effect using by anti-4-1BB antibody, the combined therapy with the other ingredients to enhance immune response against cancer-specific antigen has been considered recently.

CTX (cyclophosphamide), a chemotherapeutics to treat cancer disease is the cell proliferation inhibitor inhibiting rapidly growing cancer cell in cancer patient (Lake R A and Robinson B W, Nat. Rev. Cancer, 5(5), pp 397˜405, 2005). Since most of chemotherapeutic anti-cancer drugs inhibit cell proliferation, it has been reported to show the side effect in rapidly growing normal cell other than cancer cells (Lake R A and Robinson B W, Nat. Rev. Cancer, 5(5), pp 397˜405, 2005; Bast R C Jr et al., Clin. Immunol. Immunopathol., 28(1), pp 101˜114, 1983; Mackall C L et al., Blood, 84(7), pp 2221˜2228, 1994). The combined therapy with an antibody and chemotherapeutic anti-cancer drug has been anticipated to be ineffective thereby.

However, it has been known that CTX selectively removes only CD4⁺ CD25⁺ regulatory T cell however does not remove normal CD4 and CD8 T cell (Ghiringhelli F et al., Eur. J. Immunol., 34(2), pp 336˜344, 2004; Taieb J et al., J. Immunol., 176(5), pp 2722˜2729, 2006; Cupps T R et al., J. Immunol., 128(6), pp 2453˜2457, 1982; Winkelstein A, Immunology, 46(4), pp 827˜832, 1982; Mackall C L et al., Blood, 84(7), pp 2221˜2228, 1994). Due to those properties, it gives rise to direct removal ability of cancer cell and immune-enhancing activity therefore CTX has been regarded as an immunotherapeutic agent in spite of chemotherapeutics (Lake R A and Robbinson B W, Nat. Rev. Cancer, 5(5), pp 397˜405, 2005; Tsung K et al., J. Immunol., 160(3), pp 1369˜1377, 1998; Le H N et al., J. Immunol., 167(12), pp 6765˜6772, 2001).

The present inventors have studied to prove the improving and treating effect of combined therapy with an agonistic anti-4-1BB antibody and other chemotherapeutics including CTX on B16-F10 melanoma cancer cell line and finally discovered that the combined therapy with an anti-4-1BB antibody and anti-cancer chemotherapeutics is more effective than sole medication of anti-4-1BB antibody or chemotherapeutics for improving and treating cancer disease, which has confirmed by demonstrating the enhanced cancer cell-specific immune response and potent removing activity of cancer cell.

### SUMMARY OF THE INVENTION

The present invention also provides a use of combined mixture of anti-4-1BB antibody and chemotherapeutic anti-cancer agent for the preparation of therapeutic agent for treating or preventing cancer disease in a mammal including human in need thereof.

The present invention also provides an immunotherapeutic method for treating or preventing cancer disease comprising administering to mammal an effective amount of combined mixture of anti-4-1BB antibody and chemotherapeutic anti-cancer agent as an effective ingredient, together with a pharmaceutically acceptable carrier thereof.

### DISCLOSURE OF THE INVENTION

Accordingly, it is an object of the present invention to provide a pharmaceutical composition comprising combined mixture of anti-4-1BB antibody and chemotherapeutic anti-cancer agent enhancing the specific immune response to cancer and killing cancer cell for treating or preventing cancer disease as an effective ingredient, together with a pharmaceutically acceptable carrier.

In accordance with another aspect of the present invention, there is also provided a use of combined mixture of anti-4-1BB antibody and chemotherapeutic anti-cancer agent for the preparation of therapeutic agent for treating and preventing cancer disease in a mammal including human in need thereof.

In accordance with the other aspect of the present invention, there is also provided a method for treating or preventing cancer disease comprising administering to mammal in an effective amount of combined mixture of anti-4-1BB antibody and chemotherapeutic anti-cancer agent as an effective ingredient, together with a pharmaceutically acceptable carrier thereof.

In accordance with the other aspect of the present invention, there is also provided a method for enhancing immune response comprising administering to mammal in an effective amount of combined mixture of anti-4-1BB antibody and chemotherapeutic anti-cancer agent as an effective ingredient, together with a pharmaceutically acceptable carrier thereof. The term “anti-4-1BB antibody” disclosed herein comprise 4-1BB (CD137) molecule-specific polypeptide, preferably monoclonal anti-4-1BB antibody.

The term “4-1BB” disclosed herein comprise a 4-1BB of diverse mammal including human but does not limit thereto in the present invention.

The term “chemotherapeutic anti-cancer agent’ disclose herein comprise cyclophosphamide, cisplatin, 5-fluorouracil, irinotecan, paclitaxel or Doxorubicin, preferably, cyclophosphamide.

The pharmaceutical composition for treating or preventing cancer disease of the present invention could contain about 0.01 to 80 w/w %, preferably 0.1 to 50 w/w % of the above-described ingredients of the present invention based on the total weight of the composition.

The term “cancer disease” disclosed herein comprise lung cancer, arsenic cellular lung cancer, liver cancer, colon cancer, bone cancer, pancreatic cancer, skin cancer, cephalic or cervical cancer, skin or endophthalmic melanoma, hysterocarcinoma, ovarian cancer, rectal cancer, stomach cancer, perianal cancer, colonic cancer, breast cancer, endometrioma, cervical carcinoma, vaginal carcinoma, vulvul carcinoma, Hodgkin's disease, esophageal cancer, enteric cancer, endocrine gland cancer, thyroid cancer, parathyroid cancer, adrenal cancer, smooth tissue sarcoma, urethral cancer, penile cancer, prostatic cancer, chronic or acute leukemia, lymphocytoma, cystic cancer, nephritic or hydrouretic cancer, renal cell carcinoma, renal pelvic carcinoma, CNS tumor, primary CNS lymphoma, spinal medulla tumor, brain stem neuroglioma, hypophyseal adenomatosis and the like, preferably, lung cancer, liver cancer, skin cancer or endophthalmic melanoma.

The combined treatment of anti-4-1BB antibody and chemotherapeutic anti-cancer agent, i.e., CTX of the present invention showed more potent reducing effect on tumor size, increasing activity of the survival rate of C57BL/6 mouse injected with melanoma cancer cell line, i.e., B16F10 cell, and increasing effect on the number of CD4 and CD8 T cells expressing interferon-gamma than those of sole treatment of 4-1BB. Therefore, it has been confirmed that the combined treatment of the anti-4-1BB antibody and chemotherapeutic anti-cancer agent show more potent anti-cancer activity and potent enhancing activity of immune cell than sole treatment of 4-1BB antibody.

The inventive composition may additionally comprise appropriate carriers, adjuvants or diluents, conventionally used in the art. The appropriate carriers, adjuvants or diluents is not limited to a specific material, and can be chosen, according to the usage and application method. Appropriate diluents are listed in the written text of Remington's Pharmaceutical Science (Mack Publishing Co., Easton Pa.).

The inventive anti-4-1BB antibody can be used independently or in combination with well-known cancer drug such as taxol, cyclophosphamide, doxorubicin and the like.

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or compound varies depending on the condition and the weight of the subject, severity, drug form, route and period of administration, and may be chosen by those skilled in the art. However, in order to obtain desirable effects, it is generally recommended to administer at the amount ranging from 0.0001 to 100 mg/kg, preferably, 0.001 to 10 mg/kg by weight/day of the inventive extract of the present invention. The dose may be administered in single or divided into several times per day.

In term of composition, the inventive composition should be present between 0.01 to 80% by weight, preferably 0.5 to 50% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### BRIEF DESCRIPTION OF DRAWINGS

The above and other objects, features and other advantages of the present invention will more clearly understood from the following detailed description taken in conjunction with the accompanying drawings, in which;

FIG. 1 shows the change of tumor size (a) and the survival rate of mouse (b) treated with the combination of an anti-4-1BB and CTX, simultaneously causing to tumor by injecting B16F10 cancer cell to C57BL/6 mouse.

FIG. 2 shows the change of tumor size and survival rate of mouse, at five-days after treatment (a, b) and at 10-days after treatment (c, d) of the combination of an anti-4-1BB and CTX after causing tumor by injecting B16F10 cancer cell to C57BL/6 mouse.

FIG. 3 represents the change of tumor size and survival rate of mouse treated with the combination of an anti-4-1BB(3E1) and the other chemotherapeutics, cisplatin (a, b), 5-fluorouracil (c, d), Doxorubicin (e, f), irinotecan (g, h) and paclitaxel (i, j) after causing tumor by injecting B16F10 cancer cell to C57BL/6 mouse.

FIG. 4 represents the calculated number of lymph node cell (a), CD4 cell (b) and CD8 cell (c) of draining lymph node cell after injecting CTX and/or anti-4-1BB(3E1).

FIG. 5 presents the expressing level of IFN-γ by staining CD4 and CD8 cell in draining lymph node cell at 17 days (a) and 22 days (b) after injecting CTX and/or anti-4-1BB to C57BL/6 mouse caused tumor by injecting B16F10 cancer cell.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Reference Example 1

### Antibody and Chemical Chemotherapeutic Anti-Cancer Agent

The hybridoma producing anti-4-1BB monoclonal antibody was provided from Dr. Robert Mittler (Emory University, Atlanta, Ga.). The above antibodies were produced from the culture medium of abdominal dropsy of mouse and hybridoma and purified by using protein G-column (Sigma, St. Louis, Mo.) in laboratory. The chemotherapeutic anti-cancer agents, i.e., cisplatin (Cis) and 5-fluorouracil (5-FU) were purchased from the Choongwae Pharm. Corporation (Seoul, Korea), irinotecan from the Aventis Pharma (Seoul, Korea), paclitaxel (Taxol) from the Bristol-Myers Squibb (New York, N.Y.) and Doxorubicin (Doxo) from the Boryung Inc. (Seoul, Korea).

A purified IgG of mouse used as an antibody in control group was purchased from the Sigma-Aldrich and an anti-CD4-FITC antibody, anti-CD8a-PE antibody and anti-IFN-γ-PE antibody were purchased from the eBioscience (San Diego, Calif.).

### Reference Example 2

### Animals and Cell-Line

C57BL/6 male mice (Harlan Laboratories, Indianapolis, Ind.) were used as an experiment animal. The mice had been bred allowing freely accessible to water and feed and maintaining the temperature to 21±2° C. in 12 hours of light/dark cycle before use in test. B16F10(ATCC CRL-6475, USA), a melanoma cell line of mouse, was cultured in DMEM medium (Dulbeco's modified eagle's medium, GIBCO BRL, USA) containing 10% FBS (Fetal Bovine Serum; Gibco BRL, NY), 2 mM L-glutamine, 100 U/l penicillin (Invitrogen, USA) and 100 μg/ml streptomycin (Invitrogen, USA).

### Example 1

### Administration of an Antibody and Chemotherapeutic Anti-Cancer Agent to Mouse

4×10⁵ cells of B16F10 melanoma cell were subcutaneously injected to the back of mice to occur tumor. At the same time, 100 μg of anti-4-1BB monoclonal antibody and 3 mg of CTX were intraperitoneally injected into the mice and the antibody was further injected thereto at the interval of five days. In case of the control mice for comparing treatment time with test group, the equivalent amount of CTX and anti 4-1BB monoclonal antibody were injected in a similar method at the 5^{th} days or the 10^{th} days after casing to tumor by injecting cancer cell. Also, in case of the control mice for comparing with various chemotherapeutic anti-cancer agent, 50˜200 μg of cisplatin (Cis), 800˜10,000 μg of 5-fluorouracil (5-FU), 2 mg of irinotecan and 200˜500 μg of paclitaxel (Taxel) were intraperitoneally injected into the mice simultaneously with cancer cell treatment and 200˜400 μg of Doxorubicin (Doxo) was intraveneously injected in a similar method. The size and survival rate of the mice were periodically determined.

### Example 2

### Determination of Change of the Immune Cell in Draining Lymph Node

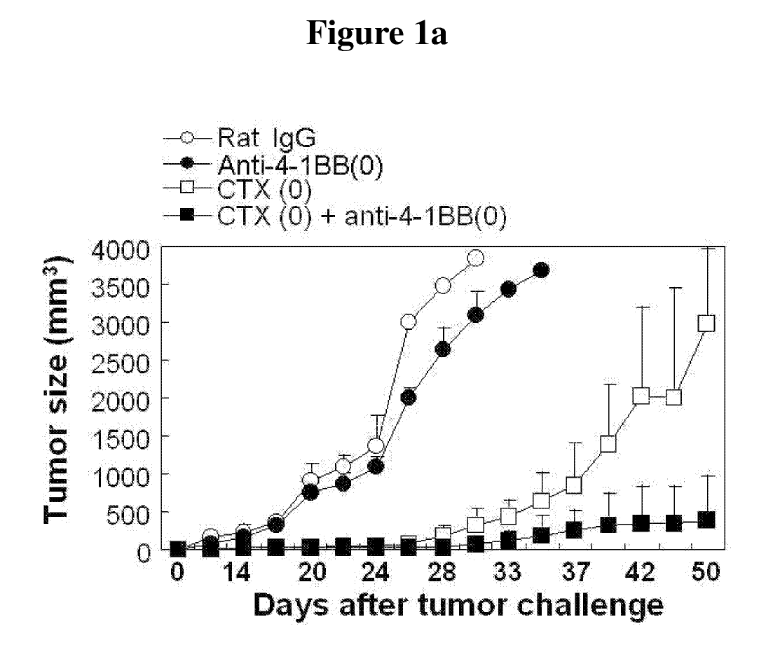

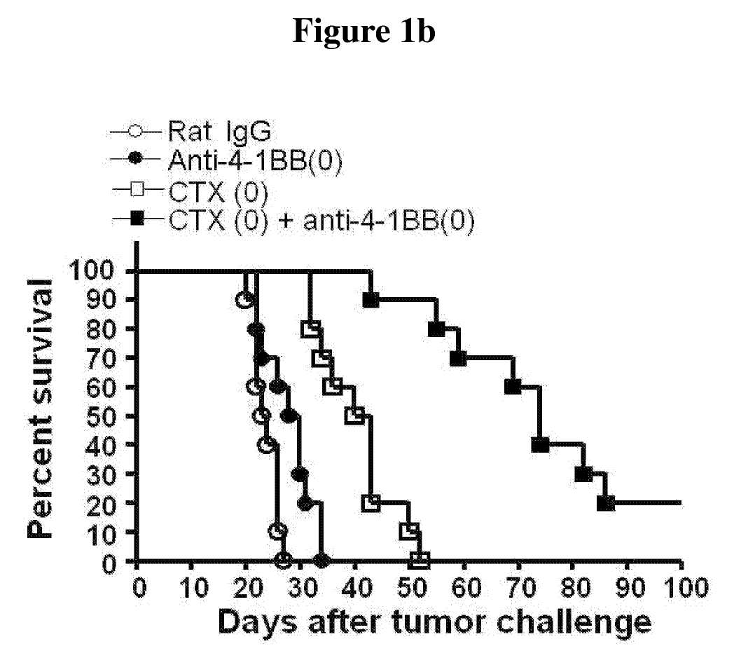

To determine the change of the immune cell in draining lymph node after treating with combination of an anti 4-1BB and anti-cancer agent, 3 mg of CTX and/or 100 μg of anti 4-1BB monoclonal antibody were injected into the peritoneal cavity once directly after causing the tumor to the mice in a similar method to Example 1 (Tsung K et al., J. Immunol., 160(3), pp 1368˜1377, 1998; Wilcox R A et al., J. Clin. Invest., 109(5), pp 651˜659, 2002). At 1^{st}, 2^{nd}, 4^{th}, 8^{th}, 12^{th}, 16^{th}, 20^{th}, 24^{th} days after the injection, the draining lymph node was isolated from the mice to count the number of the cell. To count the number of CD5 and CD8 T cells, lymph-node cell suspension was prepared from each group and the cell was reacted with Fc blocking antibody (2.4G2, BD Biosciences, USA) at 4° C. for 10 min for blocking the non-specific binding of stained antibodies through Fc region, and the surface of the cells was stained with anti CD4-FITC (eBioscience, USA) and anti-CD8a-PE antibody (eBioscience, USA). The ratio of immune cell in each sample was analyzed with FACScan (BD Bioscience, USA), and the number of infiltrated CD4+ CD8+ T cells in tumor tissue was calculated by following Math FIG. 1

$$\begin{matrix}\begin{matrix}Number of CD 4 + \\ and CD 8 +\end{matrix} \\ T cell in tumor tissue\end{matrix} = \frac{( \begin{matrix}( Number of Total Immune Cell Infiltrated in Tumor Cell ) \times \\ Ratio of CD 4 + or CD 8 + T cell ( \% )\end{matrix} )}{( 100 )} \qquad \text{Math Figure 1}$$

### Example 3

### The Expression Effect of IFN-γ in Lymph Node

To determine the expression effect of IFN-γ in lymph node caused by the combined injection of an anti 4-1BB and anti-cancer drug, 3 mg of CTX and/or 100 μg of anti 4-1BB monoclonal antibody were injected into the peritoneal cavity once directly after causing the tumor in mice in a similar method to Example 1. At 1^{st}, 2^{nd}, 4^{th}, 8^{th}, 12^{th}, 16^{th}, 20^{th}, 24^{th} days after the injection, the closest draining lymph node (inguinal lymph node) to tumor tissue in each group was isolated from the mice to determine the expression of IFN-γ (Kim Y H et al., Cell. Immunol., 238(2), pp 76˜86, 2005). To stain the IFN-γ cytokine in cell, lymph-node cell suspension was prepared from each group and the separated cells were cultured in culture medium containing Brefeldin A (BD Bioscience, USA) for 6 hours after treating with 50 ng/ml of PMA and 500 mg/ml of Ionomycin (Sigma, USA). 6 hours after the incubation, the cells were reacted with Fc blocking antibody (2.4G2, BD Biosciences, USA) at 4° C. for 10 min for blocking the non-specific binding of stained antibody through Fc region, and the surface of the cells were stained with an anti FITC-anti CD8 or anti CD4. The cells was then stained with anti-IFN-γ-PE (eBioscience, USA) using by Cytofix/cytoperm (BD Pharmingen, USA) with the manual of manufacturing company and analyzed with FACScan (BD Bioscience, USA).

### Experimental Example 1

### The Anticancer Effect of the Combined Treatment of Anti 4-1BB Monoclonal Antibody and CTX

### 1-1. The Preventing Effect of the Combined Treatment of Anti-4-1BB Antibody and CTX on Cancer

To determine the preventing effect of the combined treatment of an agonistic anti-4-1BB antibody and CTX on cancer cell, 4×10⁵ cells of B16F10 melanoma cell were injected to the back of C57BL/6 mice to induce tumor tissue in a similar method to Example 1. At the same time, 100 μg of anti-4-1BB monoclonal antibody and 3 mg of CTX were intraperitoneally injected to the mice once and anti-4-1BB monoclonal antibody was injected into the peritoneal cavity at 6 times per every 5 days. The tumor size and the survival rates of mice were analyzed during the test period (See FIG. 1)

As shown in FIG. 1, in case of mice injected with only B16F10 melanoma cancer cell line, the tumor started to grow since about 15^{th} days after the treatment and all the mice were died around 30^{th} days after injecting cancer cell. In case of mice treated with only an anti-4-1BB antibody, the growing rate of tumor did not show different with that of negative-control group however the survival days of mice were extended for 6˜7 days. In case of mice treated with only CTX, the cancer tissue was not remarkably increased by 30^{th} days so it showed potent inhibiting effect on the growth of cancer and the survival of mice were extended for 20 days. In case of mice treated with combination of an anti-4-1BB antibody and CTX, the tumor size had not been increased till 50^{th} days after injecting cancer cells and the survival rate of mice was more than 90%. 20% mice treated with the combination of anti-4-1BB antibody and CTX had survived for more than 100 days.

### 1-2. The Treating Effect of Combined Treatment of an Anti-4-1BB Antibody and CTX on Cancer

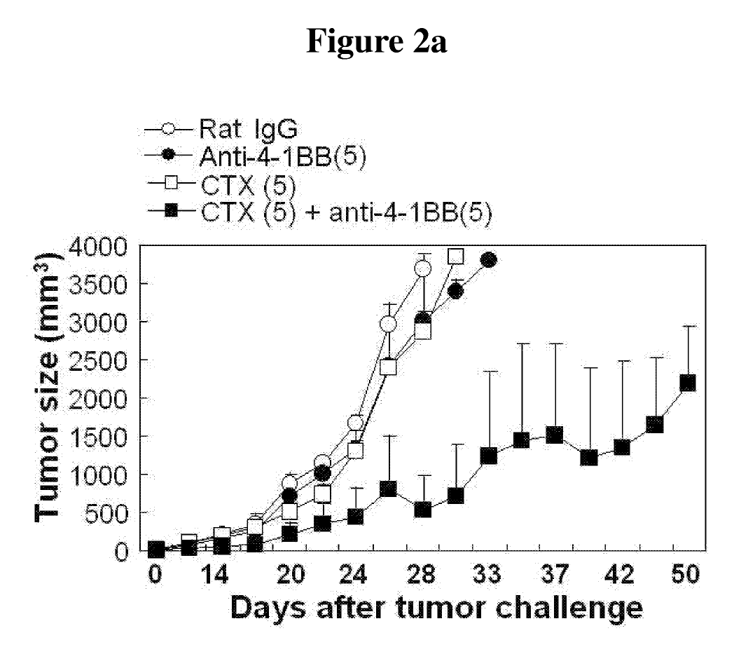

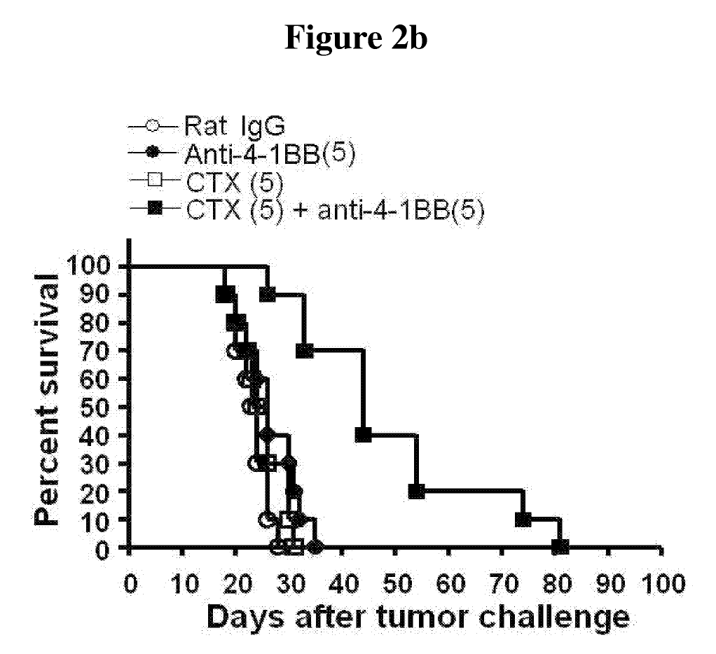

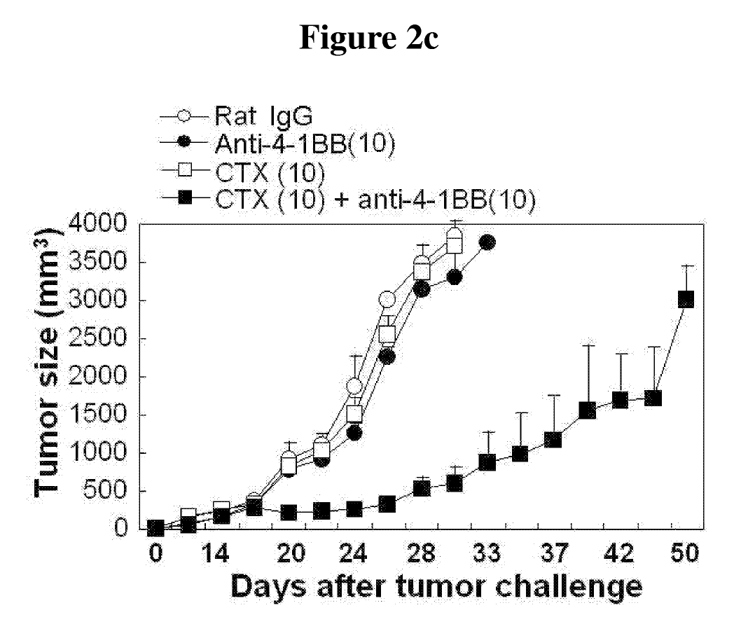

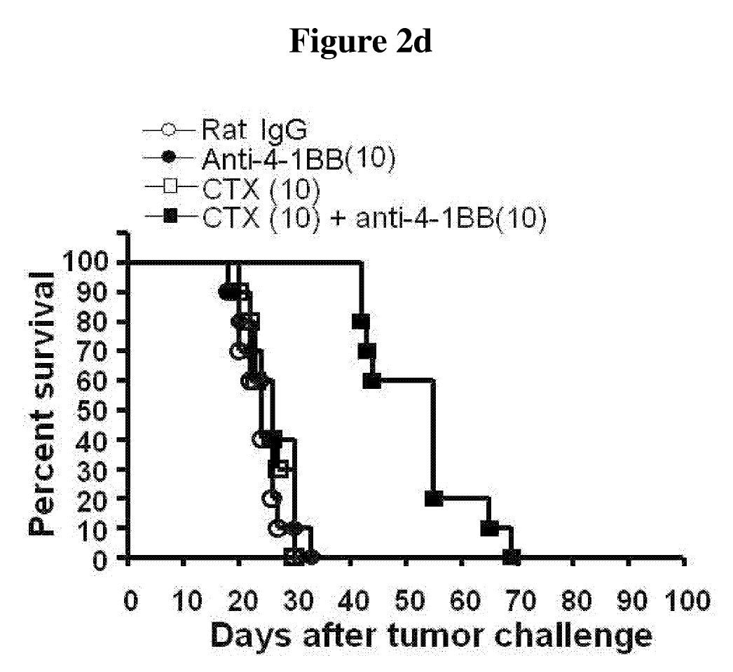

To determine the treating effect of combined treatment or sole treatment of an agonistic anti-4-1BB antibody and/or CTX, 4×10⁵ cells of B16F10 melanoma cell were injected to the back of C57BL/6 mice to induce tumor tissue in a similar method to Example 1. 3 mg of CTX were injected into the peritoneal cavity of the mice at 5^{th} days and 10^{th} days after injecting cancer cells, anti 4-1BB antibody was injected into the peritoneal cavity at 6 times per every 5 days from 5^{th} days or 10^{th} days after injecting cancer cells. The size of tumor and the survival rates of mice were analyzed during test period (See FIG. 2).

As shown in FIG. 2a and FIG. 2b, in case of mice treated with only anti-4-1BB antibody at 5^{th} days after injecting cancer cell, the growth of tumor and the survival rate of mice did not show any change. In case of mice treated with only CTX, it did not show any effect on the anti-cancer activity contrary to the result in Experimental Example 1-1. In case of mice treated with only anti-4-1BB antibody or CTX, all the mice were died within 35 days and the growth rate of cancer was similar to that of control group treated with Rat IgG. However, in case of the combined treatment with an anti-4-1BB antibody and CTX, the growth of tumor had been decreased until the 30^{th} days, and started to increase after 30^{th} day. The survival rate of the group treated with an anti-4-1BB antibody and CTX stared to decrease 30 days after injecting cancer cells, and the survival rate the group was 40% at 50^{th} days. Finally, all the mice was died at around 80^{th} day.

As shown in FIG. 2c and FIG. 2d, in case of mice treated with only anti-4-1BB antibody or CTX, 10^{th} days after injecting cancer cells, the growth of tumor and the survival of mice in the group did not show any change similarly with result of the treatment group with an anti-4-1BB antibody or CTX at 5 days after injecting cancer cell. However, in case of the combined treatment with an anti-4-1BB antibody and CTX, the growth of tumor had been decreased similarly to the result of the treatment group with only anti-4-1BB antibody and/or CTX at 5 days after injecting cancer cell, the survival rate of the group was remarkably increased.

CTX effectively induces the anti-cancer response before cancer cell forms a cancer tissue however the effect was decreased after cancer cell forms the tissue. Also, anti-4-1BB antibody could not induce sufficiently the anti-cancer response in case that the immunity of the cancer cell was low, As can be seen in the above described results, the preventing and treating effect on cancer can be increased only in case of the combined treatment with CTX and anti-4-1BB antibody.

### Experimental Example 2

### The Anticancer Effect of Combined Treatment with Anti 4-1BB Monoclonal Antibody and Chemotherapeutic Anti-Cancer Agent

To determine the preventing effect on cancer, similar to the method in Experimental Example 1, the combined treatment with an agonistic anti-4-1BB antibody and other chemotherapeutic anti-cancer agents was performed as follows. 4×10⁵ cells of B16F10 melanoma cell were injected to the back of C57BL/6 mice to occur tumor in a similar method in

### Example 1

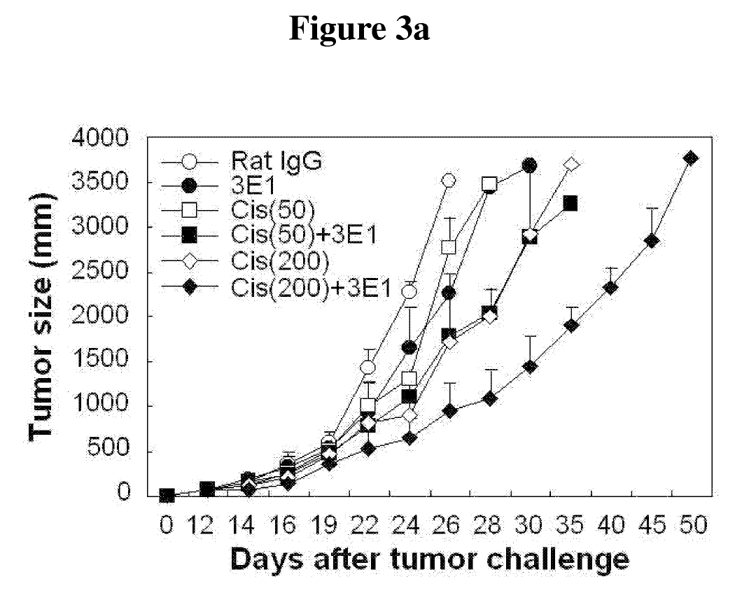

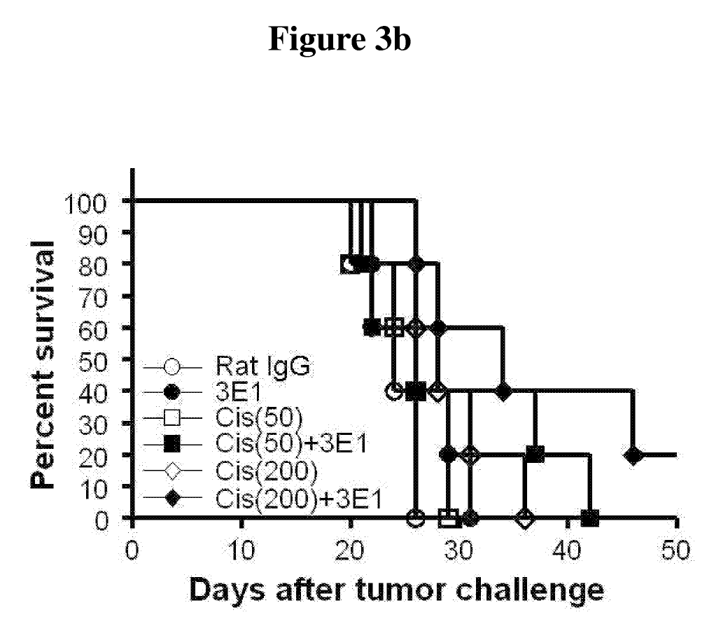

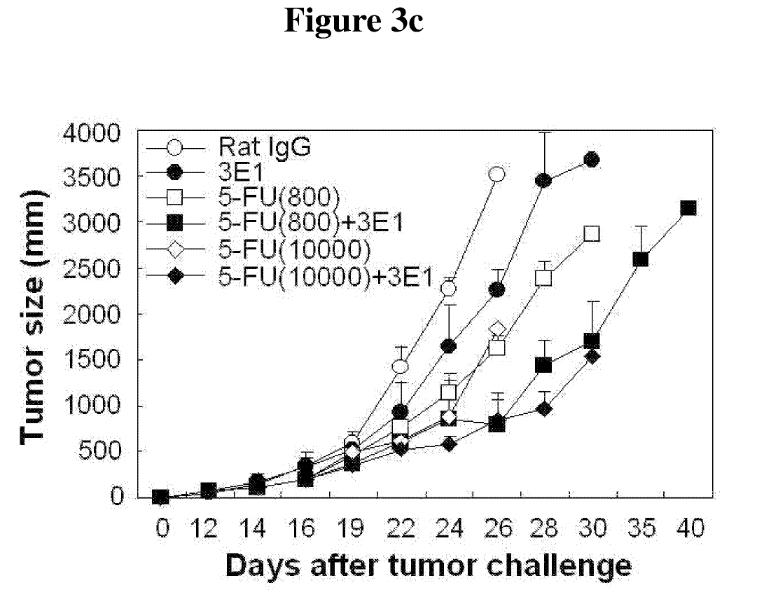

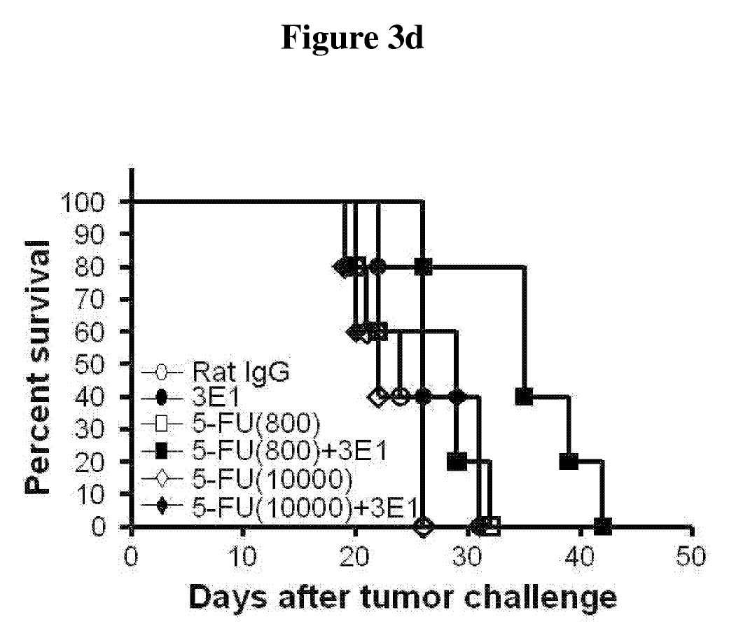

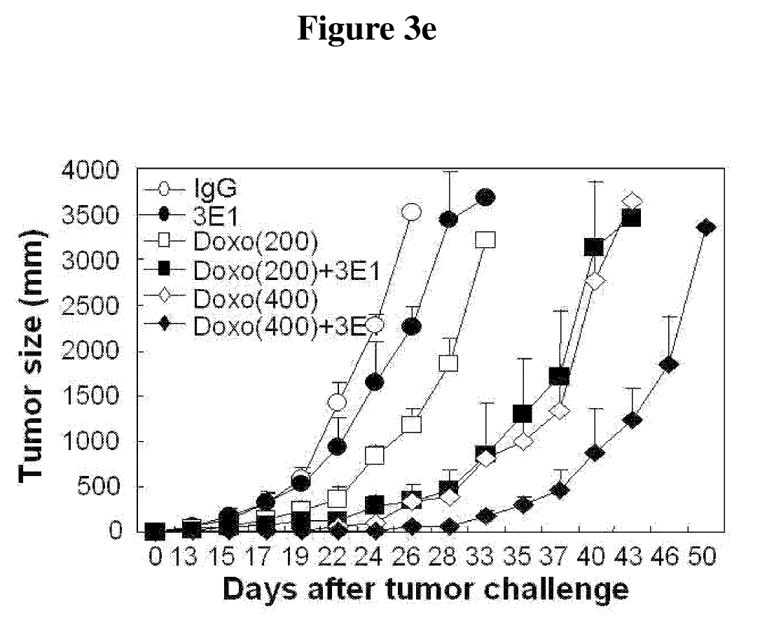

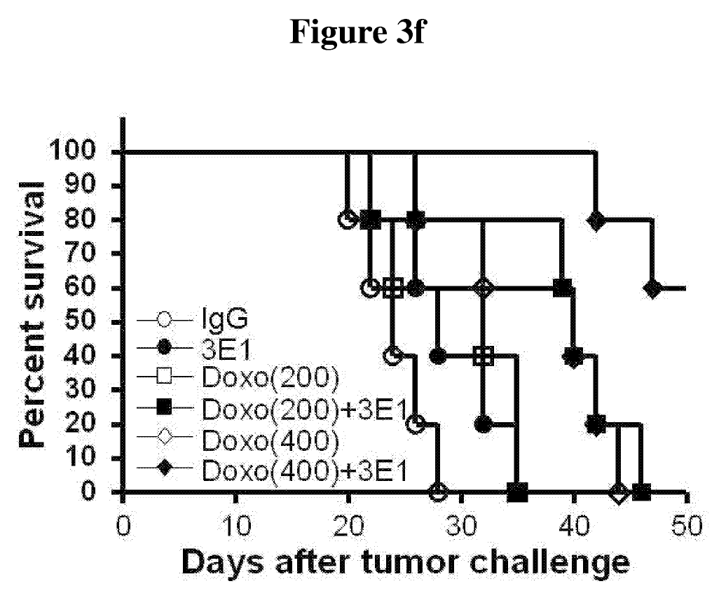

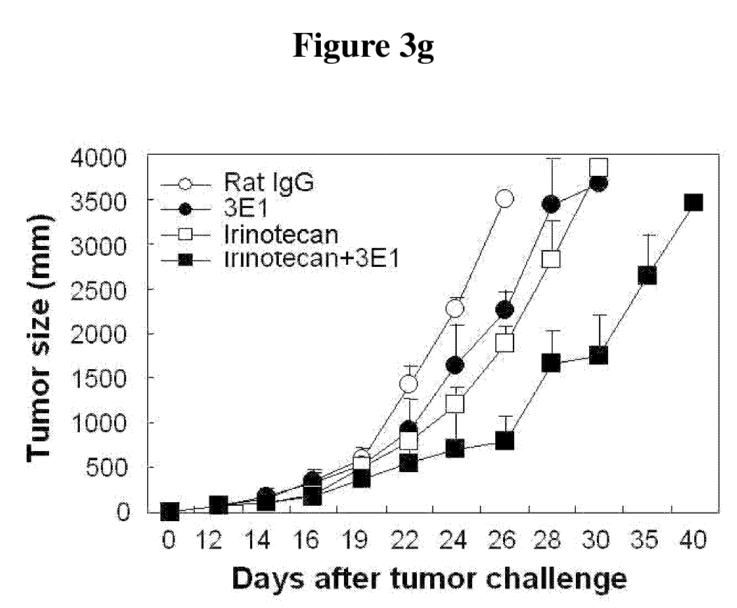

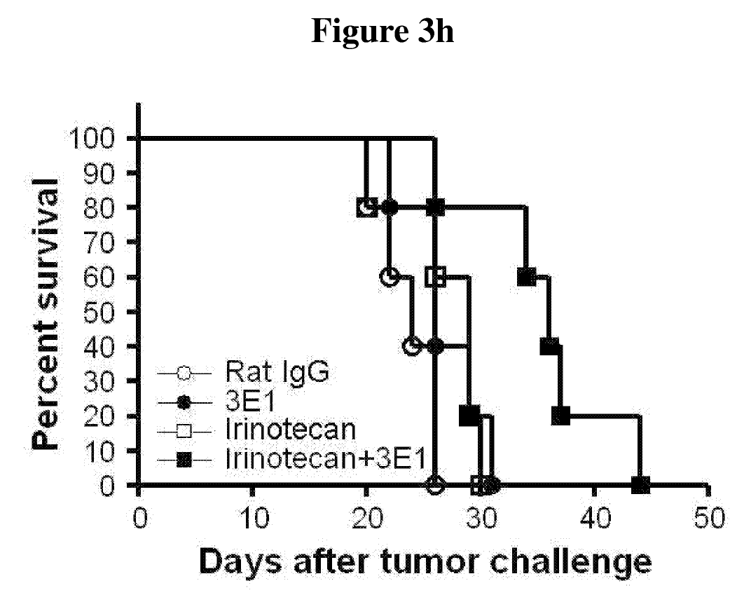

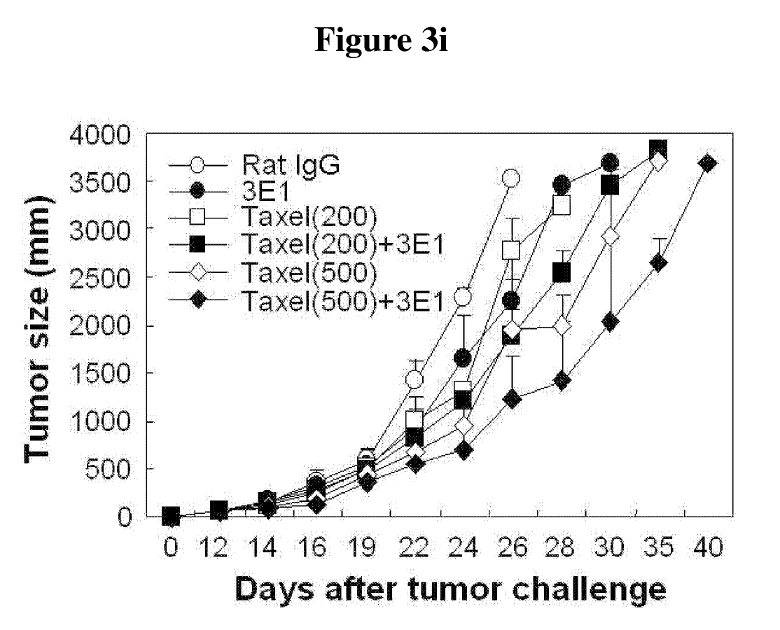

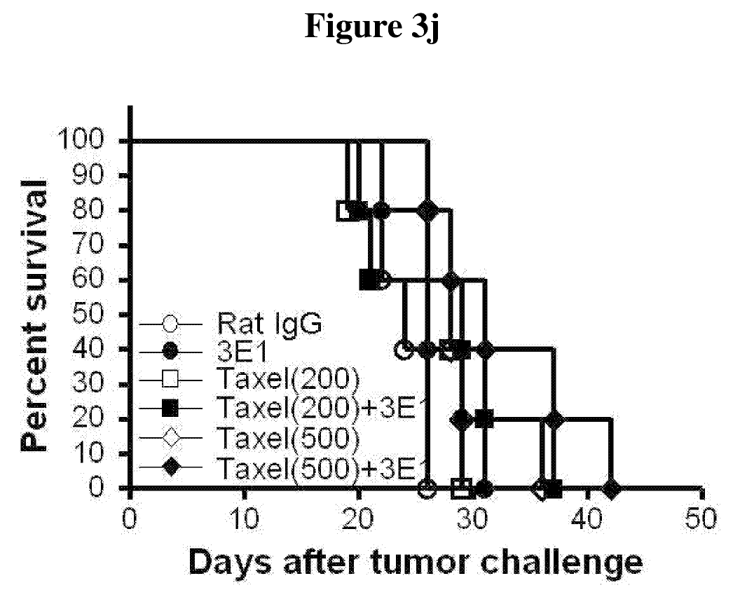

At the same time, 100 μg of anti-4-1BB monoclonal antibody and/or 50 or 200 μg of cisplatin, 800 or 10,000 μg of 5-fluorouracil (5-FU), 2 mg of irinotecan and 200 or 500 μg of paclitaxel (Taxel) were intraperitoneally injected into the mice once respectively and the anti-4-1BB monoclonal antibody was further injected into the peritoneal cavity at 6 times per every 5 days. 100 μg of anti-4-1BB monoclonal antibody and/or 200 or 400 μg of Doxorubicin (Doxo) were injected into the vein and an anti-4-1BB monoclonal antibody was injected into the peritoneal cavity at 6 times per every 5 days. The size of tumor and the survival rates of the mice were analyzed during the test period (See FIG. 3).

### 2-1. The Anticancer Effect of the Combined Treatment with Anti 4-1BB Monoclonal Antibody and Cisplatin

As shown in FIG. 3a and FIG. 3b, in case of the mice injected with only cisplatin (Cis), the growth rate of cancer tissue had been decreased in a dose dependent manner comparing with that of the control group treated with rat IgG., the survival rate of mice was partly increased. The group treated with an agonistic anti-4-1BB antibody and Cis showed more potent anticancer activity than the group treated with sole treatment. The group treated with 200 μg of Cis and anti-4-1BB antibody showed the lowest growth rate of cancer tissue and about 20% mice survived until 50^{th} day after injecting cancer cell.

### 2-2. The Anticancer Effect of the Combined Treatment of Anti 4-1BB Monoclonal Antibody with 5-Fluorouracil

As shown in FIG. 3c and FIG. 3d, in case of mice injected only 5-florouracil (5-FU), the growth rate of cancer tissue has been decreased in a dose dependent manner, whereas the survival rate of mice was increased to the contrary. In case of mice treated with an agonistic anti-4-1BB antibody and 5-FU, the growth rate of cancer tissue had been decreased and the survival rate of mice was increased.

### 2-3. The Anticancer Effect of Combined Treatment with an Anti 4-1BB Monoclonal Antibody and Doxorubicin

As shown in FIG. 3e and FIG. 3f, the group injected only Doxorubicin (Doxo) showed strong anticancer effect in a dose dependent manner, so the growth of cancer tissue had been decreased and the survival rate of mice was remarkably increased. The group treated with an agonistic anti-4-1BB antibody and Doxo showed more potent anticancer effect than the group treated with sole treatment, so in case of mice treated with 400 μg of Doxo and anti-4-1BB antibody, the growth of cancer tissue had been decreased and the survival rate of mice was maintained to 60% level of the group until 50 days after injecting cancer cell.

### 2-4. The Anticancer Effect of Combined Treatment with Anti 4-1BB Monoclonal Antibody and Irinotecan

As shown in FIG. 3g and FIG. 3h, the group injected only Irinotecan showed strong anticancer effect in a dose dependent manner and most of mice died within 30 days, of which result was similar to that of control group treated with rat IgG. The group treated with an agonistic anti-4-1BB antibody and Irinotecan showed potent synergic anticancer effect and all the mice died around 40 days.

### 2-5. The Anticancer Effect of Combined Treatment with an Anti 4-1BB Monoclonal Antibody and Paclitaxel

As shown in FIG. 3i and FIG. 3j, the group injected only Paclitaxel showed a little lower inhibiting effect of the growth of B16-F10 melanoma comparing with other anticancer agents. The growth of cancer tissue has slightly slowed and the survival rate was slightly increased in proportion to concentration however there was no significance comparing with the control group treated with rat IgG. Also, the group treated with an agonistic anti-4-1BB antibody and Paclitaxel showed the improved effect however there did not show particular significance.

### Experimental Example 3

### The Stimulating Effect of Lymphocyte Repopulation by the Combined Treatment with an Anti 4-1BB Monoclonal Antibody and CTX

There have been reported that CTX directly removes the cancer cell as well as immune cell partly. Especially, it has been reported that CTX selectively removes B cells and CD4+CD25+ T cell (Ghiringhelli F et al., Eur. J. Immunol., 34(2), pp 336˜344, 2004; Taieb J et al., J. Immunol., 176(5), pp 2722˜2729, 2006; Cupps T R et al., J. Immunol., 128(6), pp 2453˜2457, 1982; Winkelstein A, Immunology, 46(4), pp 827˜832, 1982).

Accordingly, it was anticipated that the sustainable treatment effect of cancer by the combined treatment with CTX and anti-4-1BB antibody would be involved in the repopulation of T cell showing anticancer activity. To verify the above anticipation, B16F10 melanoma cell were injected to the back of C57BL/6 mice to induce tumor tissue in a similar method to Example 1. At the same time, CTX were injected into the mice and an anti 4-1BB monoclonal antibody was injected into the peritoneal cavity per every 5 days, and the change of the cell number in lymph node was analyzed according to the method disclosed in Example 2.

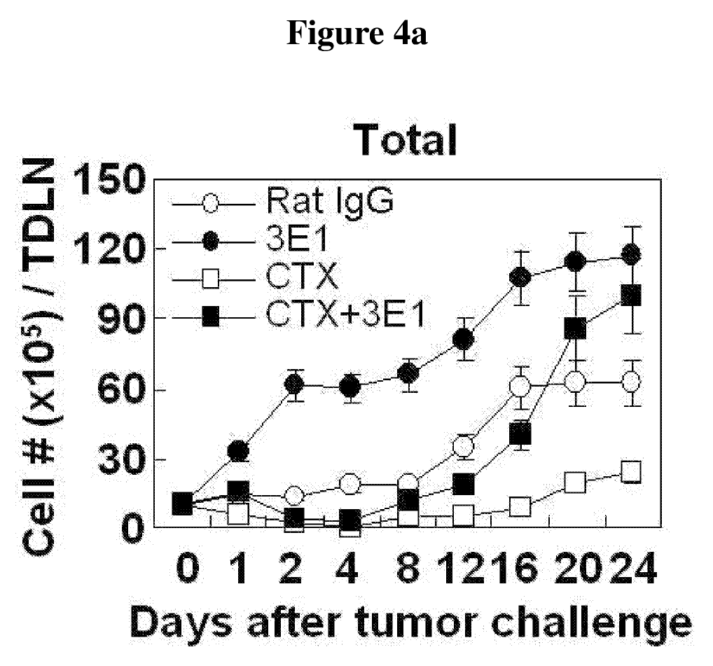

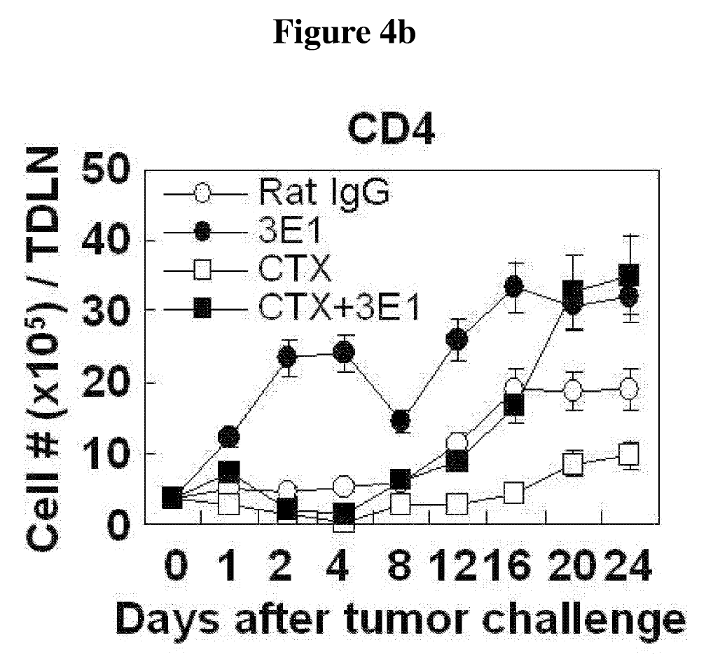

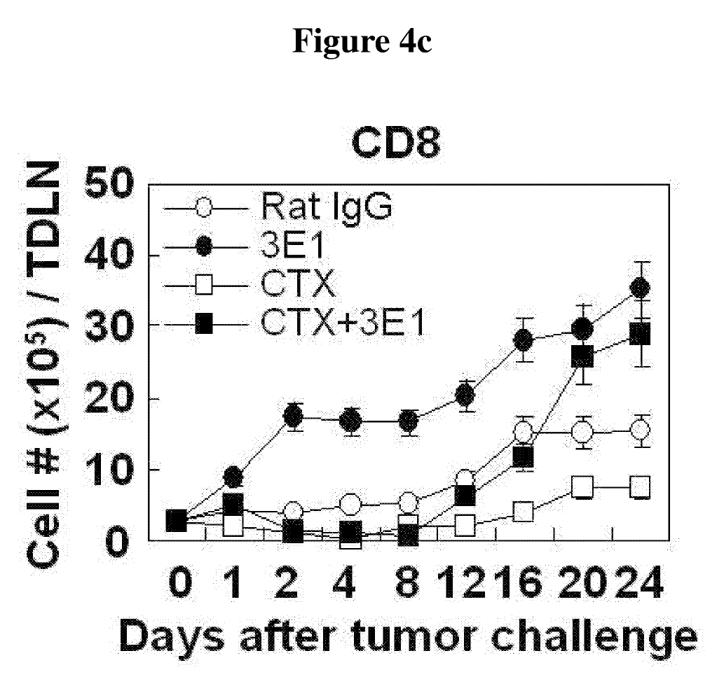

As shown in FIG. 4a, the number of cell in lymph node of mice treated with rat IgG progressively has been increased since the 8^{th} days after injecting cancer cell, and dramatically increased within 2 to 3 days in case of treatment with anti-4-1BB antibody. In case of treating CTX, the number of cell in lymph node of mice has temporarily been decreased, and increased around 16 days after injecting cancer cell, however, stayed in the level of less than ½ to ⅕ comparing with that of mice treated with rat IgG or anti-4-1BB even if the cell number was recovered thereafter. In case of the combined treatment with CTX and anti-4-1BB antibody, the number of cell in lymph node of mice was temporarily decreased however it has been increased since the 8^{th} day after injecting cancer cell. It has been recovered at the 20^{th} day similarly to the group treated with only anti-4-1BB antibody. The number of CD4 and CD8 T cell showed tendency similar to the total cell number in lymph node too (See. FIG. 4b and FIG. 4c). In case of treating anti-4-1BB antibody, the number of CD4 and CD8 T cell was dramatically increased and in case of treating CTX, the number of CD4 and CD8 T cell was decreased contrary to the above-described result. In case of sole treatment of CTX, the number of CD4 and CD8 T cell was recovered to normal range after the 14^{th} day, however in case of the combined treatment with CTX and anti-4-1BB antibody, the recovery started from the 8^{th} day. It has confirmed that in case of treating anti-4-1BB antibody, the side effect of CTX, i.e., reduced immune cell was compensated and finally, the anticancer activity could be increased by the combined treatment with an anti-4-1BB antibody and CTX.

### Experimental Example 4

### The Increasing Effect of IFN-γ Expression by the Combined Treatment with an Anti 4-1BB Monoclonal Antibody and CTX

IFN-γ plays important roles in treating cancer using by T cell, especially, it is the most representative cytokine increased by stimulating of anti-4-1BB antibody (Ikeda H et al., Cytokine Growth Factor Rev., 13(2), pp 95˜109, 2002; Ye Z et al., Nat. Med., 8(4), pp 343˜348, 2002). Accordingly, to determine whether the treating activity of cancer by the combination with an anti-4-1BB antibody and CTX is involved in the increase of IFN-γ expression or not, following experiment was performed.

3 mg of CTX and/or 100 μg of anti-4-1BB antibody were treated to the tumor-injected mice once and the cell was separated from the experimental group at 17^{th} day when the cell started to launch the repopulation after treating with CTX and at 22^{nd} day when the repopulation of the cell in lymph node was enough and the expression of IFN-γ was analyzed with FACScan (BD Bioscience, USA) with the method disclosed in Example 3.

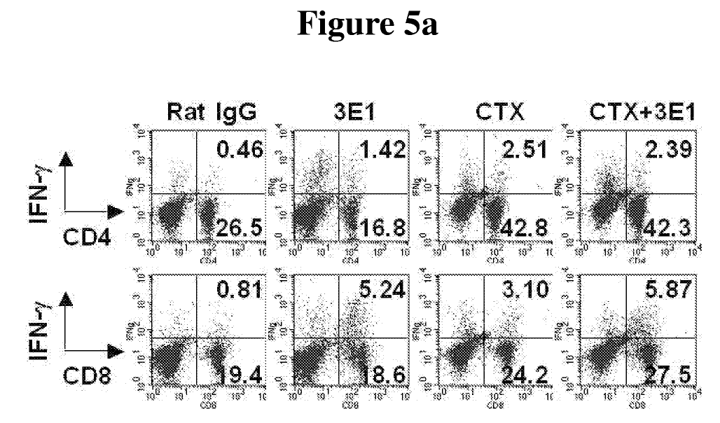

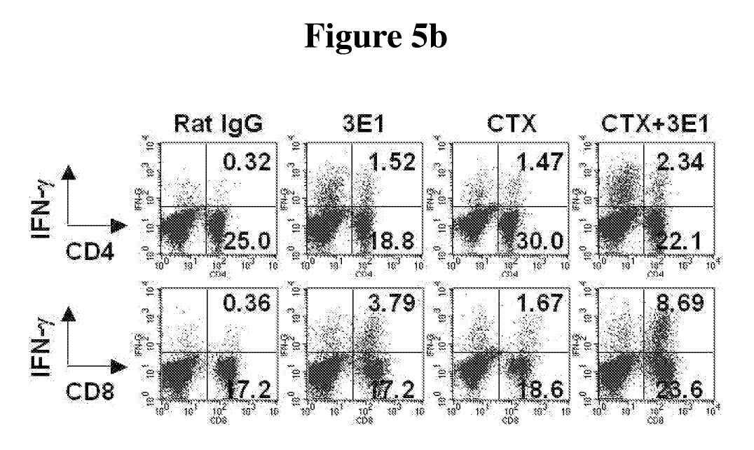

At the result of analyzing the separated cell at 17^{th} day after treating CTX, as shown in FIG. 5a, CD4 and CD8 T cell of the group treated with rat IgG expressed IFN-γ with low level (<1%) whereas the IFN-γ level, especially, CD8 T cell, was increased in case of treating anti-4-1BB antibody (about 5%). In case of treating CTX, IFN-γ expression of T cell was a little increased (2 to 3%). In case of the combined treatment with CTX and anti-4-1BB antibody, the IFN-γ expression of CD4 T cell was not dramatically changed (about 2.3%) however IFN-γ expression of CD8 T cell was increased (5 to 6%).

At the result of analyzing IFN-γ at the 22^{nd} day after treating CTX, it showed similar result to the group of treating with rat IgG, anti-4-1BB antibody or CTX however in case of the combined treatment with CTX and an anti-4-1BB antibody, about ⅓ out of CD8 T cells expressed IFN-γ (See FIG. 5b). These results postulate that the inconsistent anticancer effect of the group treated with CTX with anti-4-1BB antibody is caused by the increase of CD8 T cell expressing IFN-γ.

The above results showed that the combined treatment with an anti-4-1BB antibody and chemotherapeutic anti-cancer agent could gain synergic activity of treating cancer and the combined therapy with the drug inducing immune response and an agonistic anti-4-1BB antibody could be effective to treat the cancer cell having low antigenicity.

Hereinafter, the formulating methods and kinds of excipients will be described, but the present invention is not limited to them. The representative preparation examples were described as follows.

Preparation of injection

| | | |
|---|---|---|
| Anti-4-1BB antibody and CTX | 100 | mg |
| Sodium metabisulfite | 3.0 | mg |
| Methyl paraben | 0.8 | mg |
| Propyl paraben | 0.1 | mg |

| | |
|---|---|
| Distilled water for injection | optimum |
| | amount |

### Injection preparation was prepared by dissolving active component, controlling pH to about 7.5 and then filling all the components in 2 ml ample and sterilizing by conventional injection preparation method.

Preparation of powder

| | |
|---|---|
| Anti-4-1BB antibody and CTX | 500 mg |
| Corn Starch | 100 mg |
| Lactose | 100 mg |
| Talc | 10 mg |

### Powder preparation was prepared by mixing above components and filling sealed package.

Preparation of tablet

| | |
|---|---|
| Anti-4-1BB antibody and CTX | 200 mg |
| Corn Starch | 100 mg |
| Lactose | 100 mg |
| Magnesium stearate | optimum amount |

### Tablet preparation was prepared by mixing above components and entabletting.

Preparation of capsule

| | |
|---|---|
| Anti-4-1BB antibody and CTX | 100 mg |
| Lactose | 50 mg |
| Corn starch | 50 mg |
| Talc | 2 mg |
| Magnesium stearate | optimum amount |

### Tablet preparation was prepared by mixing above components and filling gelatin capsule by conventional gelatin preparation method.

Preparation of liquid

| | | |
|---|---|---|
| Anti-4-1BB antibody and CTX | 1000 | mg |
| Sugar | 20 | g |
| Polysaccharide | 20 | g |
| Lemon flavor | 20 | g |

### Liquid preparation was prepared by dissolving active component, and then filling all the components in 1000 ml ample and sterilizing by conventional liquid preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the combined composition comprising an anti-4-1BB antibody and chemotherapeutic anti-cancer agent of the present invention showed potent inhibiting effect on cancer cell and potent enhancing effect cancer cell-specific immune response. Accordingly, it can be useful in the prevention or treatment of cancer diseases and it could provide an immune therapy of cancer disease.

1. A pharmaceutical composition comprising combined mixture of anti-4-1BB antibody and chemotherapeutic anti-cancer agent enhancing the specific immune response to cancer and killing cancer cell for treating or preventing cancer disease as an effective ingredient, together with a pharmaceutically acceptable carrier.

2. The pharmaceutical composition of claim 1, wherein said chemotherapeutic anti-cancer agent is selected from the group consisting of cyclophosphamide, cisplatin, 5-fluorouracil, irinotecan, paclitaxel and Doxorubicin.

3. The pharmaceutical composition of claim 1, wherein said cancer disease is selected from lung cancer, arsenic cellular lung cancer, liver cancer, colon cancer, bone cancer, pancreatic cancer, skin cancer, cephalic or cervical cancer, skin or endophthalmic melanoma, hysterocarcinoma, ovarian cancer, rectal cancer, stomach cancer, perianal cancer, colonic cancer, breast cancer, endometrioma, cervical carcinoma, vaginal carcinoma, vulvul carcinoma, Hodgkin's disease, esophageal cancer, enteric cancer, endocrine gland cancer, thyroid cancer, parathyroid cancer, adrenal cancer, smooth tissue sarcoma, urethral cancer, penile cancer, prostatic cancer, chronic or acute leukemia, lymphocytoma, cystic cancer, nephritic or hydrouretic cancer, renal cell carcinoma, renal pelvic carcinoma, CNS tumor, primary CNS lymphoma, spinal medulla tumor, brain stem neuroglioma, or hypophyseal adenomatosis.

4. A use of combined mixture of anti-4-1BB antibody and chemotherapeutic anti-cancer agent for the preparation of therapeutic agent for treating and preventing cancer disease in a mammal in need thereof.

5. The use of combined mixture of anti-4-1BB antibody and chemotherapeutic anti-cancer agent of claim 4, wherein the mammal is a Human.

6. A method for treating or preventing cancer disease comprising administering to a mammal an effective amount of the combined mixture of anti-4-1BB antibody and chemical anti-cancer medicine as an effective ingredient, together with a pharmaceutically acceptable carrier thereof.

7. The method for treating or preventing cancer disease of claim 6, wherein the mammal is a Human.

8. A method for enhancing immune response comprising administering to a mammal in an effective amount of combined mixture of anti-4-1BB antibody and chemotherapeutic anti-cancer agent as an effective ingredient, together with a pharmaceutically acceptable carrier thereof.

9. The method for enhancing immune response of claim 8, wherein the mammal is a Human.
